(19) United States
(12) Patent Application Publication
(10) Pub. No.: US 2023/0240890 A1
(43) Pub. Date: Aug. 3, 2023

(54) CONTROL COMPONENT WITH FORCE FEEDBACK

(71) Applicant: ForSight Robotics Ltd., Yokneam Illit (IL)

(72) Inventors: Yoav GOLAN, Tel-Aviv (IL); Ori BEN ZEEV, Ramat Hasharon (IL); Tal KORMAN, Tel Aviv (IL); Daniel GLOZMAN, Kfar Yona (IL)

(21) Appl. No.: 18/298,490

(22) Filed: Apr. 11, 2023

Related U.S. Application Data
(63) Continuation of application No. PCT/IB2022/061636, filed on Dec. 1, 2022.
Application No. PCT/IB2022/061636 is a continuation of application No. 63/406,881, filed on Sep. 15, 2022.
(60) Provisional application No. 63/285,218, filed on Dec. 2, 2021.

(51) Int. Cl. A61F 9/007; A61B 34/00; A61B 34/20

(52) U.S. Cl. CPC A61F 9/00754; A61B 34/76; A61B 34/20; A61B 2034/2059

(57) ABSTRACT

Apparatus and methods are described for performing a procedure on a patient's eye. A robotic unit inserts an ophthalmic tool into the patient's eye via an incision in a cornea of the patient's eye. The location and the orientation of the tip of a control-component tool that is configured to be moved by an operator is determined, based upon data received from one or more location sensors that are disposed on a control-component arm that is coupled to the control-component tool, and the tip of the ophthalmic tool is moved within the patient's eye in a manner that corresponds with movement of the control-component tool. Force feedback is provided to the operator via the control-component arm. Other applications are also described.

### CROSS-REFERENCES TO RELATED APPLICATIONS

The present application is a continuation of PCT application No. PCT/IB2022/061636 to Golan, filed Dec. 1, 2022, entitled “Force feedback for robotic microsurgical procedures”, which claims priority from U.S. Provisional Patent Application No. 63/285,218 to Korman, filed Dec. 2, 2021, entitled “Robotic unit for microsurgical procedures”, and from U.S. Provisional Patent Application No. 63/406,881 to Golan, filed Sep. 15, 2022, entitled “Force feedback for robotic microsurgical procedures”. Both of the aforementioned US Provisional applications are incorporated herein by reference.

### FIELD OF EMBODIMENTS OF THE INVENTION

Some applications of the present invention generally relate to medical apparatus and methods. Specifically, some applications of the present invention relate to apparatus and methods for performing microsurgical procedures in a robotic manner.

### BACKGROUND

Cataract surgery involves the removal of the natural lens of the eye that has developed an opacification (known as a cataract), and its replacement with an intraocular lens. Such surgery typically involves a number of standard steps, which are performed sequentially.

In an initial step, the patient's face around the eye is disinfected (typically, with iodine solution), and their face is covered by a sterile drape, such that only the eye is exposed. When the disinfection and draping has been completed, the eye is anesthetized, typically using a local anesthetic, which is administered in the form of liquid eye drops. The eyeball is then exposed, using an eyelid speculum that holds the upper and lower eyelids open. One or more incisions (and typically two or three incisions) are made in the cornea of the eye. The incision(s) are typically made using a specialized blade, which is called a keratome blade. At this stage, lidocaine is typically injected into the anterior chamber of the eye, in order to further anesthetize the eye. Following this step, a viscoelastic injection is applied via the corneal incision(s). The viscoelastic injection is performed in order to stabilize the anterior chamber and to help maintain eye pressure during the remainder of the procedure, and also in order to distend the lens capsule.

In a subsequent stage, known as capsulorhexis, a part of the anterior lens capsule is removed. Various enhanced techniques have been developed for performing capsulorhexis, such as laser-assisted capsulorhexis, zepto-rhexis (which utilizes precision nano-pulse technology), and marker-assisted capsulorhexis (in which the cornea is marked using a predefined marker, in order to indicate the desired size for the capsule opening).

Subsequently, it is common for a fluid wave to be injected via the corneal incision, in order to dissect the cataract's outer cortical layer, in a step known as hydrodissection. In a subsequent step, known as hydrodelineation, the outer softer epi-nucleus of the lens is separated from the inner firmer endo-nucleus by the injection of a fluid wave. In the next step, ultrasonic emulsification of the lens is performed, in a process known as phacoemulsification. The nucleus of the lens is broken initially using a chopper, following which the outer fragments of the lens are broken and removed, typically using an ultrasonic phacoemulsification probe. Further typically, a separate tool is used to perform suction during the phacoemulsification. When the phacoemulsification is complete, the remaining lens cortex (i.e., the outer layer of the lens) material is aspirated from the capsule. During the phacoemulsification and the aspiration, aspirated fluids are typically replaced with irrigation of a balanced salt solution, in order to maintain fluid pressure in the anterior chamber. In some cases, if deemed to be necessary, then the capsule is polished. Subsequently, the intraocular lens (IOL) is inserted into the capsule. The IOL is typically foldable and is inserted in a folded configuration, before unfolding inside the capsule. At this stage, the viscoelastic is removed, typically using the suction device that was previously used to aspirate fluids from the capsule. If necessary, the incision(s) is sealed by elevating the pressure inside the bulbus oculi (i.e., the globe of the eye), causing the internal tissue to be pressed against the external tissue of the incision, such as to force closed the incision.

### SUMMARY

In accordance with some applications of the present invention a robotic system is configured for use in a microsurgical procedure, such as intraocular surgery. Typically, the robotic system includes one or more robotic units (which are configured to hold tools), in addition to an imaging system, one or more displays and a control-component unit (for example, a control-component unit that includes a pair of control components, such as joysticks), via which one or more operators (e.g., healthcare professionals, such as a physician and/or a nurse) are able to control the robotic units. Typically, the robotic system includes one or more computer processors, via which components of the system and operator(s) operatively interact with each other. The scope of the present application includes mounting one or more robotic units in any of a variety of different positions with respect to each other.

Typically, movement of the robotic units (and/or control of other aspects of the robotic system) is at least partially controlled by one or more operators (e.g., healthcare professionals, such as a physician and/or a nurse). For example, the operator may receive images of the patient's eye and the robotic units and/or tools disposed therein, via the display. Based on the received images, the operator typically performs steps of the procedure. For some applications, the operator provides commands to the robotic units via the control-component unit. Typically, such commands include commands that control the position and/or orientation of tools that are disposed within the robotic units, and/or commands that control actions that are performed by the tools. For example, the commands may control a blade, a phacoemulsification tool (e.g., the operation mode and/or suction power of the phacoemulsification tool), and/or injector tools (e.g., which fluid (e.g., viscoelastic fluid, saline, etc.) should be injected, and/or at what flow rate). Alternatively or additionally, the operator may input commands that control the imaging system (e.g., the zoom, focus, and/or x-y positioning of the imaging system). For some applications, the commands include controlling an intraocular-lens-manipulator tool, for example, such that the tool manipulates the intraocular lens inside the eye for precise positioning of the intraocular lens within the eye.

Typically, the control-component unit includes one or more control-component joysticks that are configured to correspond to respective robotic units of the robotic system. For example, the system may include first and second robotic units, and the control-component unit may include first and second joysticks. Typically, each of the joysticks is a control-component arm that includes a plurality of links that are coupled to each other via joints. (The terms “joystick” and “control-component arm” are used interchangeably in the present disclosure.) For some applications, the control-component joysticks comprise respective control-component tools therein (in order to replicate the robotic units). Typically, the computer processor determines the XYZ location and orientation of the tip of the control-component tool, and drives the robotic unit such that the tip of the actual tool that is being used to perform the procedure tracks the movements of the tip of the control-component tool. In some cases, the actual tool that is being used to perform the procedure is described herein, in the specification and in the claims, as an “ophthalmic tool.” This term is used in order to distinguish the tool that is being used to perform the procedure from the control-component tool, and should not be interpreted as limiting the type of tool that may be used in any way. The term “ophthalmic tool” should be interpreted to include any one the tools described herein and or any other types of tools that may occur to a person of ordinary skill in the art upon reading the present disclosure.

Typically during a cataract procedure, one or more incisions (and typically two or three incisions) are made in the cornea of the eye. The incision(s) are typically made using a specialized blade, which is called a keratome blade. Typically, the robotic unit is configured to insert the ophthalmic tools into the patient's eye such that entry of the tool into the patient's eye is via an incision in the cornea, and the tip of the tool is disposed within the patient's eye. Further typically, the robotic system is configured to move the tip of the tool within the patient's eye in such a manner that entry of the tool into the patient's eye is constrained to remain within the incision.

In order to perform non-robotic anterior ophthalmic surgery, a surgeon typically makes one or more incisions in the patient's cornea, which is thereafter used as an entry point for various surgical tools. A tool is inserted through an incision, and is manipulated within the eye to achieve the surgical goals. While this manipulation occurs, it is medically preferable that the tool does not forcefully press against the incision edges, lift upwards, or depress downwards exceedingly. Such motions may cause tearing at the incision edges, which widens the incision and can negatively impact the surgical outcome. Ideally, the surgeon will manipulate a tool such that at the entry point of the tool through the incision, the tool is rotated about the center of the incision and not moved laterally, with such motion of the tool at the incision being described herein as maintaining the center of motion. For robotic procedures, such as those described herein, the above-described motion of the tool is described as maintaining a remote center of motion, since the tool is typically controlled from a distance (e.g., via the control-component unit). In non-robotic procedures, it can be difficult to manually maintain a center of motion, especially when the surgeon needs to focus on the tool tip, which is performing the current surgical action. In accordance with some applications of the present invention with some applications of the present invention, feedback is provided to assist an operator performing robotic-assisted ophthalmic surgery. The feedback, which is typically provided by the control-component unit (as described in further detail hereinbelow), typically assists the operator in maintaining the remote center of motion of the ophthalmic tool by applying forces that oppose the operator's attempted movements of the joysticks and/or control-component tools that would result in violation of the remote center of motion.

As described hereinabove, for some applications, the operator provides commands to the robotic units via the control-component unit. Typically, such commands include commands that control the position and/or orientation of tools that are disposed within the robotic units, and/or commands that control actions that are performed by the tools. For some applications, the robotic units are configured to allow entry of the tool into the patient's eye to move within the incision, and the computer processor is configured to drive an output unit to provide feedback to the operator that is indicative of a location of the entry of the tool into the patient's eye within the incision. For example, the computer processor may generate an output on a display that shows the incision zone and the location of entry of the tool within the incision zone. For some applications, as the tool is moved in such a manner that the location of the entry of the tool into the patient's eye is within a given distance of the edge of the incision, an output, such as a visual or audio alert, is generated.

For some applications, the computer processor is configured to drive the control-component unit to provide feedback to the operator that is indicative of a location of the entry of the tool into the patient's eye within the incision. For example, as the tool is moved in such a manner that the entry location of the tool into the patient's eye is closer to the edge of the incision, resistance to movement of the control-component arm may be increased, and/or the control-component arm may be vibrated, and/or a different output may be generated. It is noted that in accordance with some such applications of the present invention, motion of the ophthalmic tool itself is not constrained to maintain the remote center of motion. Rather, the tool is allowed to move freely, but the control-component unit provides force feedback (and/or other feedback) to the operator that is such as to assist the operator in moving the joysticks and the control-component tools in a manner that will cause the ophthalmic tool to maintain its remote center of motion location within the incision or within the incision zone.

There is therefore provided, in accordance with some applications of the present invention, apparatus for performing a procedure on an eye of a patient using an ophthalmic tool that has a tip, the apparatus including:

a robotic unit configured to move the ophthalmic tool; and

a control-component unit that includes:
a control-component tool that is configured to be moved by an operator and that defines a tip; and
at least one control-component arm coupled to the control-component tool and including one or more location sensors and;

a computer processor configured to:
drive the robotic unit to insert the ophthalmic tool into the patient's eye via an incision in a cornea of the patient's eye, such that a tip of the ophthalmic tool is disposed within the patient's eye and a remote center of motion location of the ophthalmic tool is disposed within the incision;
determine the location and the orientation of the tip of the control-component tool based upon data received from the one or more location sensors;
move the tip of the ophthalmic tool within the patient's eye in a manner that corresponds with movement of the control-component tool; and
provide feedback to the operator that is indicative of a disposition of the remote center of motion location of the ophthalmic tool relative to the incision.

In some applications, the control-component arm includes a plurality of links that are coupled to each other via rotational arm joints, and the one or more location sensors include:

three rotary encoders, each of the three rotary encoders coupled to a respective one of the rotational arm joints and configured to detect movement of the respective rotational arm joint and to generate rotary-encoder data indicative of an XYZ location of the tip of the control-component tool, in response thereto; and

an inertial measurement unit including at least one of sensor selected from the group consisting of: a three-axis accelerometer, a three-axis gyroscope, and a three-axis magnetometer, the inertial measurement unit being configured to generate inertial-measurement-unit data indicative of an orientation of the tip of control-component tool.

In some applications, the control-component arm includes a plurality of links that are coupled to each other via rotational arm joints, and the control-component tool is coupled to the control-component arm via three rotational tool joints, and the one or more location sensors include:

two rotary encoders coupled to each one of the rotational arm joints and configured to detect movement of the rotational arm joint and to generate rotary-encoder data indicative of an XYZ location of the tip of the control-component tool, in response thereto; and

one rotary encoder coupled to each one of the rotational tool joints and configured to detect movement of the rotational tool joint and to generate rotary-encoder data indicative of an orientation of the tip of the control-component tool, in response thereto;

an inertial measurement unit including at least one of sensor selected from the group consisting of: a three-axis accelerometer, a three-axis gyroscope, and a three-axis magnetometer, the inertial measurement unit being configured to generate inertial-measurement-unit data indicative of an orientation of the tip of control-component tool.

In some applications, the computer processor is configured to provide feedback to the operator that is indicative of the disposition of the remote center of motion location of the ophthalmic tool relative to the incision by generating an alert as the ophthalmic tool is moved in such a manner that the remote center of motion location of the ophthalmic tool is within a given distance from the edge of the incision.

In some applications, the computer processor is configured to generate an audio alert.

In some applications, the computer processor is configured to generate a visual alert.

In some applications, the computer processor is configured to provide feedback to the operator that is indicative of the disposition of the remote center of motion location of the ophthalmic tool relative to the incision by providing force feedback to the operator via the control-component arm.

In some applications, the computer processor is configured to:

determine an identity of the ophthalmic tool that has been inserted into the patient's eye, and

based upon the identity of the ophthalmic tool, calculate a disposition of the remote center of motion location of the ophthalmic tool relative to the incision.

In some applications, the computer processor is configured to provide force feedback to the operator via the control component, by:

performing velocity measurements on the control-component tool,

calculating a force to be applied to the operator based on the velocity measurements, and

driving the control component to apply the calculated force to the operator.

In some applications, the computer processor is configured to provide force feedback to the operator via the control-component arm, by:

performing measurements of a position of the ophthalmic tool relative to the incision,

calculating a force to be applied to the operator based on the position measurements, and

driving the control-component arm to apply the calculated force to the operator.

In some applications, the computer processor is configured to calculate a force to be applied to the operator by calculating the force such as to be equal and opposite to a force applied to the control-component tool by the operator.

In some applications, the computer processor is configured to calculate a force to be applied to the operator by calculating the force such as to be proportional to a distance of an outer edge of the ophthalmic tool from a center of the incision.

In some applications, the computer processor is configured to receive an input from the operator that is indicative of a stiffness of force feedback that they wish to receive, and to calculate a force to be applied to the operator at least partially based upon the input from the operator.

In some applications, the computer processor is configured to constrain movement of the control-component tool in a manner that corresponds to how movement of the remote center of motion location of the ophthalmic tool relative to the incision should be constrained.

In some applications, the computer processor is configured to constrain movement of the control-component tool in a manner that constrains the remote center of motion location of the ophthalmic tool to remain within an incision zone that is larger than the incision.

In some applications, the computer processor is configured to constrain movement of the control-component tool in a manner that constrains the remote center of motion location of the ophthalmic tool to remain within the incision.

In some applications, the computer processor is configured to calculate a force to be applied to the operator by calculating a force function that is based on a distance of an outer edge of the ophthalmic tool from a center of the incision in two directions.

In some applications, a first one of the two directions is parallel to the incision and at a tangent to the cornea of the patient's eye at the incision, and a second one of the two directions is normal to the first direction and at a tangent to the cornea of the patient's eye at the incision.

In some applications:

the control-component arm includes a plurality of links that are coupled to each other via rotational arm joints, and one or more motors that are operatively coupled to respective rotational arm joints; and

the computer processor is configured to provide force feedback to the operator by driving the control-component arm using the plurality of motors.

In some applications, the control-component arm includes exactly three motors operatively coupled to respective joints.

In some applications, the control-component arm includes a belt, and at least one of the motors is operatively coupled to a corresponding one of the rotational arm joints via the belt, such that the at least one of the motors is disposed closer to a base of the control-component unit than if the at least one of the motors directly drove the corresponding one of the rotational arm joints.

In some applications, a majority of the one or more motors directly drive a corresponding one of the rotational arm joints to which they are operatively coupled.

In some applications, the one or more location sensors includes:

three rotary encoders, each of the three rotary encoders coupled to a respective one of the rotational arm joints and configured to detect movement of the respective rotational arm joint and to generate rotary-encoder data indicative of an XYZ location of the tip of the control-component tool, in response thereto; and

an inertial measurement unit including at least one of sensor selected from the group consisting of: a three-axis accelerometer, a three-axis gyroscope, and a three-axis magnetometer, the inertial measurement unit being configured to generate inertial-measurement-unit data indicative of an orientation of the tip of control-component tool.

In some applications, the control-component tool is coupled to the control-component arm via three rotational tool joints, and the one or more location sensors include:

two rotary encoders coupled to each one of the rotational arm joints and configured to detect movement of the rotational arm joint and to generate rotary-encoder data indicative of an XYZ location of the tip of the control-component tool, in response thereto; and

one rotary encoder coupled to each one of the rotational tool joints and configured to detect movement of the rotational tool joint and to generate rotary-encoder data indicative of an orientation of the tip of the control-component tool, in response thereto;

an inertial measurement unit including at least one of sensor selected from the group consisting of: a three-axis accelerometer, a three-axis gyroscope, and a three-axis magnetometer, the inertial measurement unit being configured to generate inertial-measurement-unit data indicative of an orientation of the tip of control-component tool.

There is further provided, in accordance with some applications of the present invention, apparatus for performing a procedure on an eye of a patient using an ophthalmic tool that has a tip, the apparatus including:

a robotic unit configured to move the tool;

a control-component unit that includes:
a control-component tool that is configured to be moved by an operator and that defines a tip; and
a control-component arm coupled to the control-component tool and including:
   a plurality of links that are coupled to each other via rotational arm joints;
   one or more location sensors; and
   one or more motors that are operatively coupled to respective rotational arm joints:

a computer processor configured to:
drive the robotic unit to insert the ophthalmic tool into the patient's eye via an incision in a cornea of the patient's eye, such that a tip of the ophthalmic tool is disposed within the patient's eye;
determine a location and orientation of the tip of the control-component tool based upon data received from the one or more locations sensors;
move the tip of the selected ophthalmic tool within the patient's eye in a manner that corresponds with movement of the control-component tool; and
provide force feedback to the operator by driving the control-component arm using the plurality of motors.

In some applications, the control component includes exactly three motors operatively coupled to respective rotational arm joints.

In some applications, the control-component arm includes a belt, and at least one of the motors is operatively coupled to a corresponding one of the rotational arm joints via the belt, such that the at least one of the motors is disposed closer to a base of the control-component unit than if the at least one of the motors directly drove the corresponding one of the rotational arm joints.

In some applications, a majority of the one or more motors directly drive a corresponding one of the rotational arm joints to which they are operatively coupled.

In some applications, the one or more location sensors include:

three rotary encoders, each of the three rotary encoders coupled to a respective one of the rotational arm joints and configured to detect movement of the respective rotational arm joint and to generate rotary-encoder data indicative of an XYZ location of the tip of the control-component tool in response thereto; and

an inertial measurement unit including at least one of sensor selected from the group consisting of: a three-axis accelerometer, a three-axis gyroscope, and a three-axis magnetometer, the inertial measurement unit being configured to generate inertial-measurement-unit data indicative of an orientation of the tip of control-component tool.

In some applications, the control-component tool is coupled to the control-component arm via three rotational tool joints, and the one or more location sensors include:

two rotary encoders coupled to each one of the rotational arm joints and configured to detect movement of the rotational arm joint and to generate rotary-encoder data indicative of an XYZ location of the tip of the control-component tool, in response thereto; and

one rotary encoder coupled to each one of the rotational tool joints and configured to detect movement of the rotational tool joint and to generate rotary-encoder data indicative of an orientation of the tip of the control-component tool, in response thereto;

an inertial measurement unit including at least one of sensor selected from the group consisting of: a three-axis accelerometer, a three-axis gyroscope, and a three-axis magnetometer, the inertial measurement unit being configured to generate inertial-measurement-unit data indicative of an orientation of the tip of control-component tool.

In some applications, the computer processor is configured to:

drive the robotic unit to insert the ophthalmic tool into the patient's eye via the incision in the cornea of the patient's eye, such that the tip of the ophthalmic tool is disposed within the patient's eye and a remote center of motion location of the ophthalmic tool is disposed within the incision; and

provide force feedback to the operator that is indicative of a disposition of the remote center of motion location of the ophthalmic tool relative to the incision.

In some applications, the computer processor is configured to:

determine an identity of the ophthalmic tool that has been inserted into the patient's eye, and

based upon the identity of the ophthalmic tool, calculate a disposition of the remote center of motion location of the ophthalmic tool relative to the incision.

In some applications, the computer processor is configured to provide force feedback to the operator via the control component, by:

performing velocity measurements on the control-component tool,

calculating a force to be applied to the operator based on the velocity measurements, and

driving the control component to apply the calculated force to the operator, via the one or more motors.

In some applications, the computer processor is configured to provide force feedback to the operator via the control component, by:

performing measurements of a position of the ophthalmic tool relative to the incision,

calculating a force to be applied to the operator based on the position measurements, and

driving the control component to apply the calculated force to the operator.

In some applications, the computer processor is configured to calculate a force to be applied to the operator by calculating the force such as to be equal and opposite to a force applied to the control-component tool by the operator.

In some applications, the computer processor is configured to calculate a force to be applied to the operator by calculating the force such as to be proportional to a distance of an outer edge of the ophthalmic tool from a center of the incision.

In some applications, the computer processor is configured to receive an input from the operator that is indicative of a stiffness of force feedback that they wish to receive, and to calculate a force to be applied to the operator at least partially based upon the input from the operator.

In some applications, the computer processor is configured to constrain movement of the control-component tool in a manner that corresponds to how movement of the remote center of motion location of the ophthalmic tool relative to the incision should be constrained.

In some applications, the computer processor is configured to constrain movement of the control-component tool in a manner that constrains the remote center of motion location of the ophthalmic tool to remain within an incision zone that is larger than the incision.

In some applications, the computer processor is configured to constrain movement of the control-component tool in a manner that constrains the remote center of motion location of the ophthalmic tool to remain within the incision.

In some applications, the computer processor is configured to calculate a force to be applied to the operator by calculating a force function that is based on a distance of an outer edge of the ophthalmic tool from a center of the incision in two directions.

In some applications, a first one of the two directions is parallel to the incision and at a tangent to the cornea of the patient's eye at the incision, and a second one of the two directions is normal to the first direction and at a tangent to the cornea of the patient's eye at the incision.

There is further provided in accordance with some applications of the present invention, apparatus for performing a procedure on an eye of a patient using a plurality of ophthalmic tools each of which has a tip, the apparatus including:

a robotic unit configured to move the ophthalmic tools; and

a computer processor configured to:
drive the robotic unit to insert a selected one of the ophthalmic tools into the patient's eye via an incision in a cornea in the patient's eye, such that a tip of the selected ophthalmic tool is disposed within the patient's eye and a remote center of motion location of the ophthalmic tool is disposed within the incision;
determine an identity of the ophthalmic tool that has been inserted into the patient's eye;
based upon the identity of the selected ophthalmic tool, calculate a disposition of the remote center of motion location of the ophthalmic tool relative to the incision; and
provide feedback to an operator that is indicative of a disposition of the remote center of motion location of the selected ophthalmic tool relative to the incision.

There is further provided in accordance with some applications of the present invention, a method for performing a procedure on an eye of a patient using an ophthalmic tool that has a tip, the method including:

driving a robotic unit to insert the ophthalmic tool into the patient's eye via an incision in a cornea of the patient's eye, such that a tip of the ophthalmic tool is disposed within the patient's eye and a remote center of motion location of the ophthalmic tool is disposed within the incision;

determining the location and the orientation of the tip of a control-component tool that is configured to be moved by an operator, based upon data received from one or more location sensors that are disposed on a control-component arm that is coupled to the control-component tool;

moving the tip of the ophthalmic tool within the patient's eye in a manner that corresponds with movement of the control-component tool; and

providing feedback to the operator that is indicative of a disposition of the remote center of motion location of the ophthalmic tool relative to the incision.

There is further provided in accordance with some applications of the present invention, a method for performing a procedure on an eye of a patient using an ophthalmic tool that has a tip, the method including:

driving a robotic unit to insert the ophthalmic tool into the patient's eye via an incision in a cornea of the patient's eye, such that a tip of the ophthalmic tool is disposed within the patient's eye and a remote center of motion location of the ophthalmic tool is disposed within the incision;

determining the location and the orientation of the tip of a control-component tool that is configured to be moved by an operator, based upon data received from one or more location sensors that are disposed on a control-component arm that is coupled to the control-component tool;

moving the tip of the ophthalmic tool within the patient's eye in a manner that corresponds with movement of the control-component tool; and

providing force feedback to the operator via the control-component arm, the control component arm includes a plurality of links that are coupled to each other via rotational arm joints and one or more motors that are operatively coupled to respective rotational arm joints and the force feedback is provided to the operator by driving the control-component arm using the plurality of motors.

There is further provided in accordance with some applications of the present invention, a method for performing a procedure on an eye of a patient using a plurality of ophthalmic tools each of which has a tip, the method including:

driving a robotic unit to insert the ophthalmic tool into the patient's eye via an incision in a cornea of the patient's eye, such that a tip of the ophthalmic tool is disposed within the patient's eye and a remote center of motion location of the ophthalmic tool is disposed within the incision;

determining an identity of the ophthalmic tool that has been inserted into the patient's eye;

based upon the identity of the selected ophthalmic tool, calculating a disposition of the remote center of motion location of the ophthalmic tool relative to the incision; and

providing feedback to the operator that is indicative of a disposition of the remote center of motion location of the selected ophthalmic tool relative to the incision.

There is further provided in accordance with some applications of the present invention, apparatus for performing robotic microsurgery on an eye of a patient using one or more tools, the apparatus including:

an end effector;

a tool mount coupled to the end effector and configured to securely hold the one or more tools;

one or more robotic arms coupled to the end effector and which are configured to control yaw and pitch angular rotations of the one or more tools, such that a tip of a tool that is held by the tool mount is moved in a desired manner within the patient's eye, while a location of entry of the tool into the patient's eye is maintained within an incision zone that is more than 150 percent of a maximum cross section of the tool that passes through the incision zone;

a control component configured to be moved by an operator such as to move the tool in the desired manner; and

an output unit configured to provide feedback to the operator that is indicative of a location of the location of entry of the tool into the patient's eye within the incision zone.

In some applications, the output unit includes a display that shows the incision zone and the location of entry of the tool within the incision zone.

In some applications, the output unit includes an output unit that is configured to generate an alert as the tool is moved in such a manner that the location of the entry of the tool into the patient's eye is close to the edge of the incision zone.

In some applications, the output unit includes a portion of the control component that is configured to provide haptic feedback to the operator.

In some applications, the control component is configured to increase resistance to movement of the control component as the location of the entry of the tool into the patient's eye is closer to the edge of the incision zone.

The present invention will be more fully understood from the following detailed description of embodiments thereof, taken together with the drawings, in which:

### BRIEF DESCRIPTION OF THE DRAWINGS

FIG. 1 is a schematic illustration of a robotic system that is configured for use in a microsurgical procedure, such as intraocular surgery, in accordance with some applications of the present invention;

FIG. 2 is a schematic illustration of an incision in a patient's cornea, in accordance with some applications of the present invention;

FIGS. 3A and 3B are schematic illustrations of a tool inserted through a patient's cornea, such that a tip of the tool is moved in a desired manner within the patient's eye, while a location of entry of the tool into the patient's eye is maintained within an incision zone, in accordance with some applications of the present invention;

FIG. 4A, 4B, 4C, 4D, 4E, 4F, 4G, 4H, and 41 are graphs that graphically illustrate the force of the feedback that is applied to an operator by a control-component unit, as a function of the distance of a portion of the tool from the center of an incision in a subject's cornea, in accordance with respective applications of the present invention;

FIG. 5 is a flowchart showing steps of a procedure, in accordance with some applications of the present invention;

FIGS. 6A, 6B, and 6C are schematic illustrations of a joystick and control-component tool, in accordance with some applications of the present invention; and

FIG. 7 is a schematic illustration of some additional components of control-component joystick, in accordance with some applications of the present invention.

### DETAILED DESCRIPTION OF EMBODIMENTS

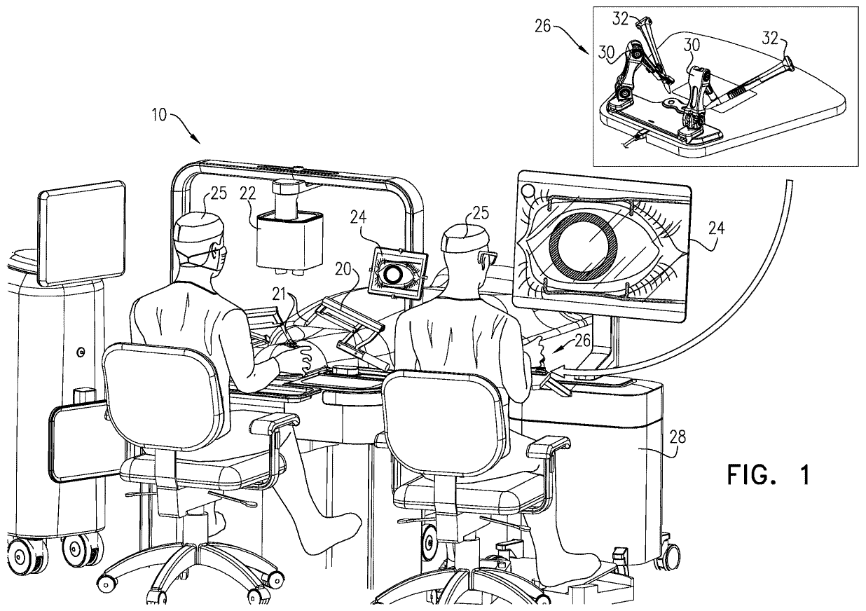

Reference is now made to FIG. 1, which is a schematic illustration of a robotic system 10 that is configured for use in a microsurgical procedure, such as intraocular surgery, in accordance with some applications of the present invention. Typically, when used for intraocular surgery, robotic system 10 includes one or more robotic units 20 (which are configured to hold tools 21), in addition to an imaging system 22, one or more displays 24 and a control-component unit 26 (e.g., control-component unit that includes a pair of control components, such as joysticks 30, as shown in the enlarged portion of FIG. 1), via which one or more operators 25 (e.g., healthcare professionals, such as a physician and/or a nurse) is able to control robotic units 20. Typically, robotic system 10 includes one or more computer processors 28, via which components of the system and operator(s) 25 operatively interact with each other. The scope of the present application includes mounting one or more robotic units in any of a variety of different positions with respect to each other.

Typically, movement of the robotic units (and/or control of other aspects of the robotic system) is at least partially controlled by one or more operators 25 (e.g., healthcare professionals, such as a physician and/or a nurse). For example, the operator may receive images of the patient's eye and the robotic units and/or tools disposed therein, via display 24. Typically, such images are acquired by imaging system 22. For some applications, imaging system 22 is a stereoscopic imaging device and display 24 is a stereoscopic display. Based on the received images, the operator typically performs steps of the procedure. For some applications, the operator provides commands to the robotic units via control-component unit 26. Typically, such commands include commands that control the position and/or orientation of tools that are disposed within the robotic units, and/or commands that control actions that are performed by the tools. For example, the commands may control a blade, a phacoemulsification tool (e.g., the operation mode and/or suction power of the phacoemulsification tool), and/or injector tools (e.g., which fluid (e.g., viscoelastic fluid, saline, etc.) should be injected, and/or at what flow rate). Alternatively or additionally, the operator may input commands that control the imaging system (e.g., the zoom, focus, and/or x-y positioning of the imaging system). For some applications, the commands include controlling an intraocular-lens-manipulator tool, for example, such that the tool manipulates the intraocular lens inside the eye for precise positioning of the intraocular lens within the eye.

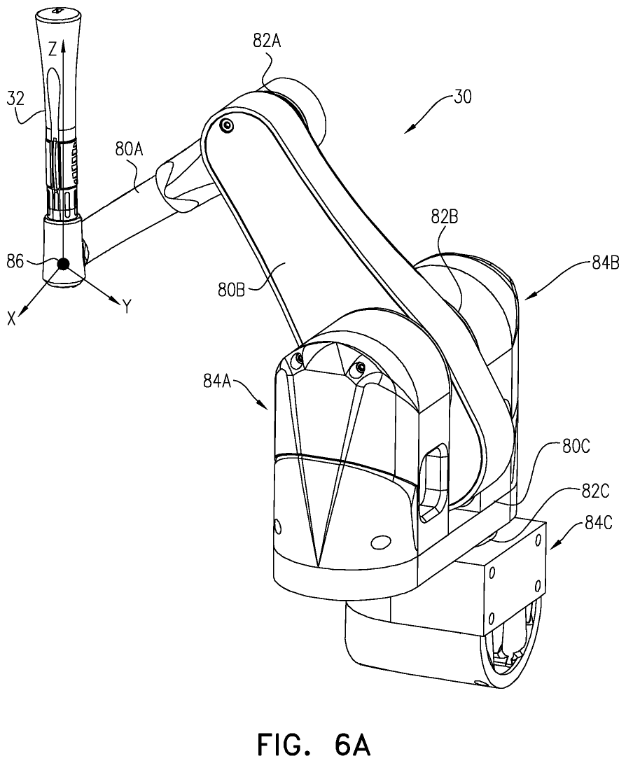

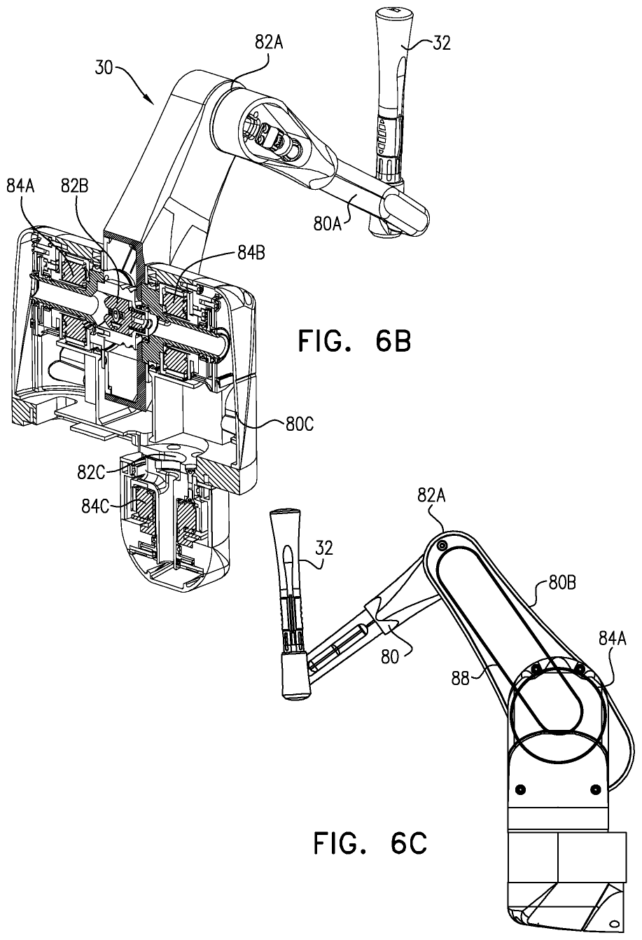

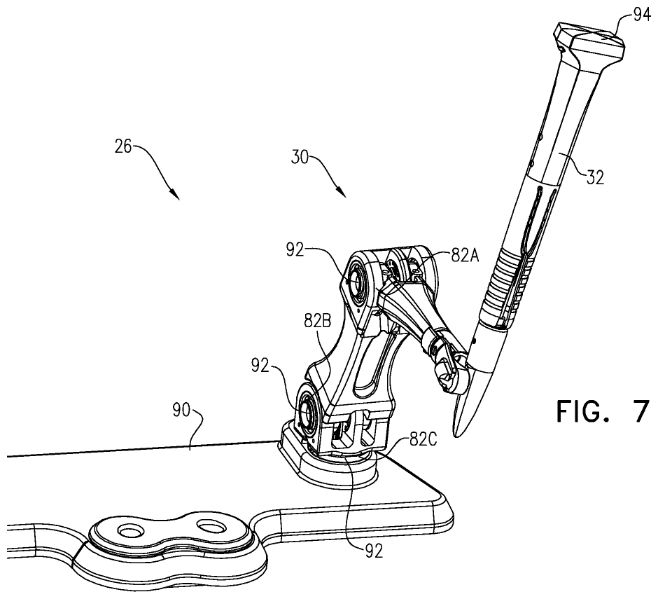

Typically, the control-component unit includes one or more control-component joysticks 30 that are configured to correspond to respective robotic units 20 of the robotic system. For example, as shown, the system may include first and second robotic units, and the control-component unit may include first and second joysticks, as shown. Typically, each of the joysticks is a control-component arm that includes a plurality of links that are coupled to each other via joints, as described in further detail hereinbelow with reference to FIGS. 6A-7. For some applications, the control-component joysticks comprise respective control-component tools 32 therein (in order to replicate the robotic units), as shown in FIG. 1. Typically, the computer processor determines the XYZ location and orientation of the tip of the control-component tool 32, and drives the robotic unit such that the tip of the actual tool 21 that is being used to perform the procedure tracks the movements of the tip of the control-component tool. In some cases, tool 21 is described herein, in the specification and in the claims, as an “ophthalmic tool.” This term is used in order to distinguish tool 21 from control-component tool 32, and should not be interpreted as limiting the type of tool that may be used as tool 21 in any way. The term “ophthalmic tool” should be interpreted to include any one the tools described herein and or any other types of tools that may occur to a person of ordinary skill in the art upon reading the present disclosure.

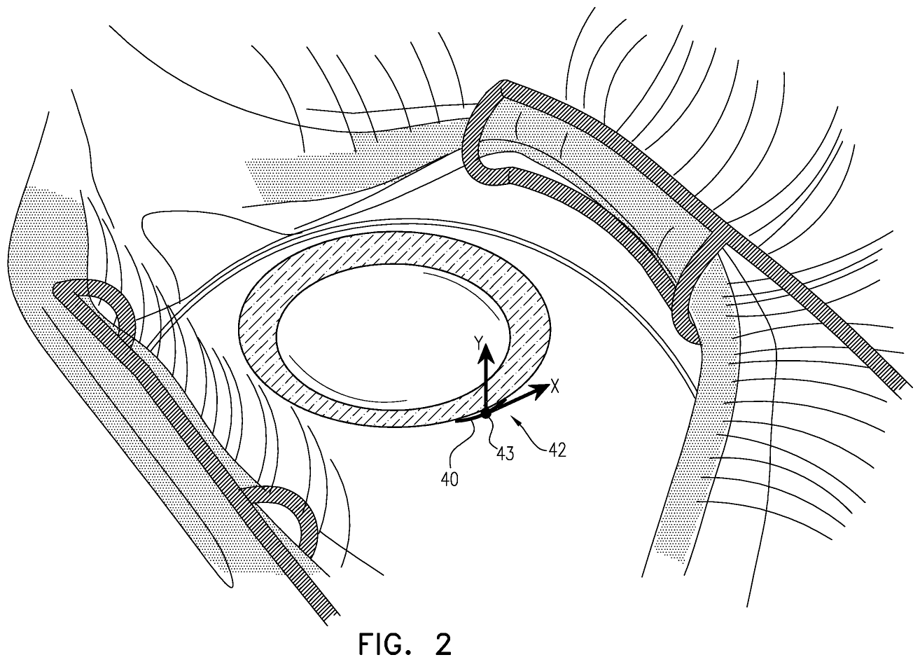

Reference is now made to FIG. 2, which is a schematic illustration of an incision 40 in a patient's cornea 42, in accordance with some applications of the present invention. As described hereinabove in the Background section, typically during a cataract procedure, one or more incisions (and typically two or three incisions) are made in the cornea of the eye. The incision(s) are typically made using a specialized blade, which is called a keratome blade. Typically, the robotic unit is configured to insert tools 21 into the patient's eye such that entry of the tool into the patient's eye is via incision 40, and the tip of the tool is disposed within the patient's eye. Further typically, robotic system 10 is configured to move the tip of the tool within the patient's eye in such a manner that entry of the tool into the patient's eye is constrained to remain within the incision. For some applications, the incision width is equal to the width of the keratome blade. The incision center point 43 (which is marked in FIG. 2) is hereby defined as the point on the corneal surface that is centered within the incision widthwise. In FIG. 2, axes have been added, with the x axis parallel to the incision and at a tangent to the cornea at the incision, and the y axis normal to the x axis, and at a tangent to the cornea at the incision. Examples of the present invention will be described hereinbelow with reference to the x and y axes.

In order to perform non-robotic anterior ophthalmic surgery, a surgeon typically makes one or more incisions in the patient's cornea, which is thereafter used as an entry point for various surgical tools. A tool is inserted through an incision, and is manipulated within the eye to achieve the surgical goals. While this manipulation occurs, it is medically preferable that the tool does not forcefully press against the incision edges, lift upwards, or depress downwards exceedingly. Such motions may cause tearing at the incision edges, which widens the incision and can negatively impact the surgical outcome. Ideally, the surgeon will manipulate a tool such that at the entry point of the tool through the incision, the tool is rotated about the center of the incision and not moved laterally, with such motion of the tool at the incision being described herein as maintaining the center of motion. For robotic procedures, such as those described herein, the above-described motion of tool 21 is described as maintaining a remote center of motion, since the tool is typically controlled from a distance (via control-component unit 26). In non-robotic procedures, it can be difficult to manually maintain a center of motion, especially when the surgeon needs to focus on the tool tip, which is performing the current surgical action. In accordance with some applications of the present invention, feedback is provided to assist an operator performing robotic-assisted ophthalmic surgery. The feedback, which is typically provided by control-component unit 26 (as described in further detail hereinbelow), typically assists the operator in maintaining the remote center of motion of tool 21 by applying forces that oppose the operator's attempted movements of joysticks 30 and/or control-component tool 32 that would result in violation of the remote center of motion.

As described hereinabove, for some applications, the operator provides commands to the robotic units via control-component unit 26 (shown in FIG. 1). Typically, such commands include commands that control the position and/or orientation of tools that are disposed within the robotic units, and/or commands that control actions that are performed by the tools. For some applications, the robotic units are configured to allow entry of the tool into the patient's eye to move within the incision, and the computer processor is configured to drive an output unit to provide feedback to the operator that is indicative of a location of the entry of the tool into the patient's eye within the incision. For example, the computer processor may generate an output on display(s) 24 that shows the incision zone and the location of entry of the tool within the incision zone. For some applications, as the tool is moved in such a manner that the location of the entry of the tool into the patient's eye is within a given distance of the edge of the incision, an output, such as a visual or audio alert, is generated. For some applications, the computer processor is configured to drive the control-component unit to provide feedback to the operator that is indicative of a location of the entry of the tool into the patient's eye within the incision. For example, as the tool is moved in such a manner that the entry location of the tool into the patient's eye is closer to the edge of the incision, resistance to movement of the control-component arm may be increased, and/or the control-component arm may be vibrated, and/or a different output may be generated. It is noted that in accordance with some such applications of the present invention, motion of tool 21 itself is not constrained to maintain the remote center of motion. Rather, the tool is allowed to move freely, but the control component provides force feedback (and/or other feedback) to the operator that is such as to assist the operator in moving the joysticks 30 and the control-component tools 32 in a manner that will cause tool 21 to maintain its remote center of motion within the incision or within the incision zone. Some applications of the above-described feedback are now described in further detail.

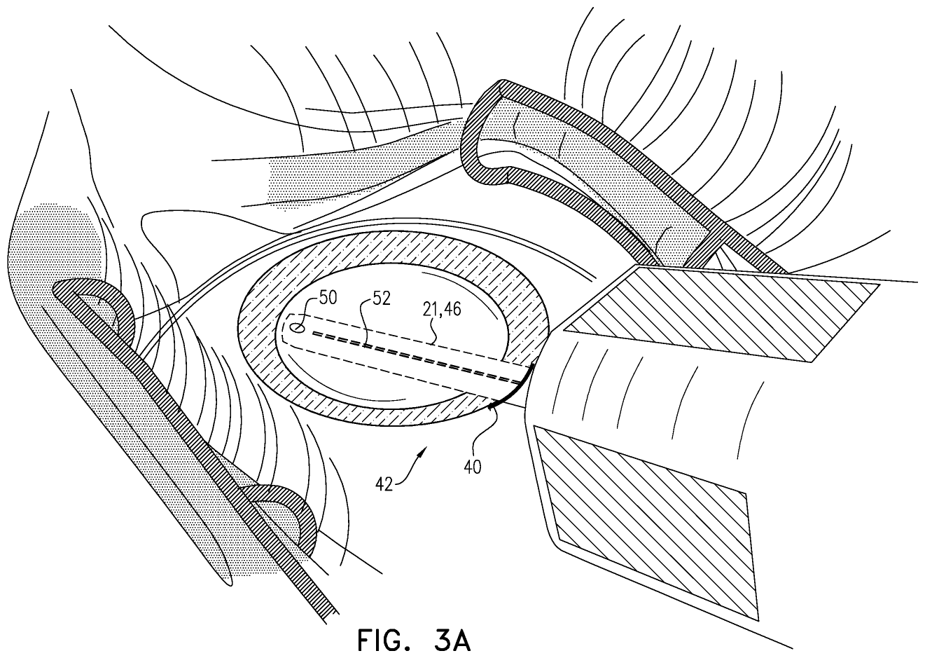

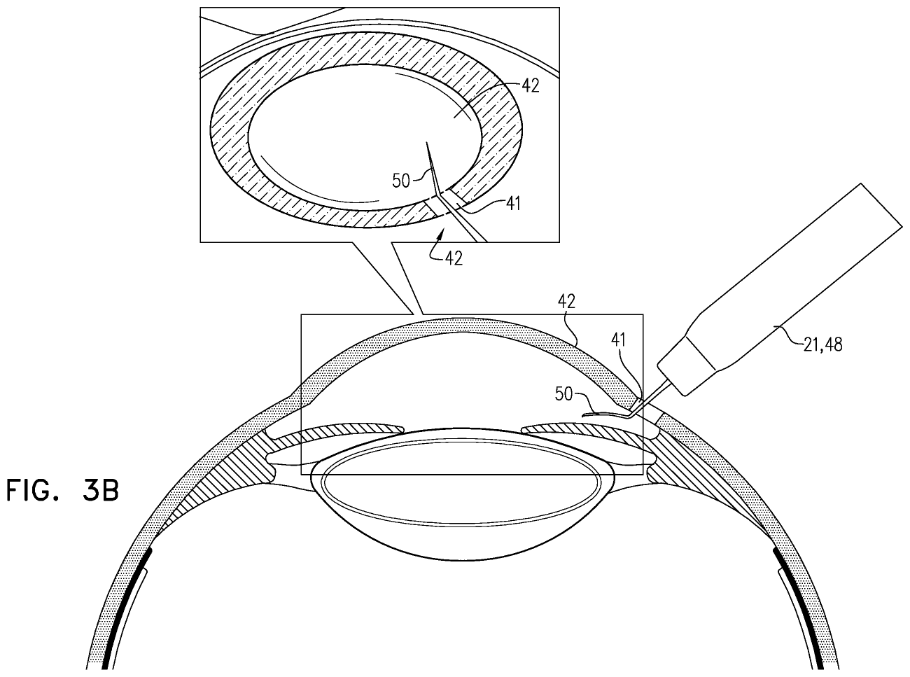

Reference is now made to FIGS. 3A and 3B, which are schematic illustrations of a tool 21 inserted through a patient's cornea 42, such that a tip 50 of the tool is moved in a desired manner within the patient's eye, while a location of entry of the tool into the patient's eye is maintained within an incision, in accordance with some applications of the present invention. FIG. 3A shows the insertion of an irrigation-aspiration tool 46 via incision 40, while FIG. 3B shows a syringe 48 being inserted. Typically, robotic system 10 is configured to insert tools 21 into the patient's eye such that entry of the tool into the patient's eye (i.e., the remote center of motion location of the tool) is via incision 40, and the tip 50 of the tool is disposed within the patient's eye. Further typically, the robotic system is configured to assist the operator in moving the tip of the tool within the patient's eye in such a manner that entry of the tool into the patient's eye (i.e., the remote center of motion location of the tool) remains within the incision. For some applications, the robotic system is configured to assist the operator by constraining movement of control-component tool in a manner that corresponds to how movement of the ophthalmic tool should be constrained, in order to prevent the entry of the tool into the patient's eye moving outside the incision. For some applications, the computer processor provides feedback to the operator that constrains movement of a portion of the control-component tool that corresponds to the portion of tool 21 that is currently within the incision (i.e., the remote center of motion location of the tool), while allowing the tip of the control-component tool (which corresponds to the tip of tool 21) to move in the desired manner. Typically, the control-component tool is configured to provide feedback using one or more control-component motors, as described in further detail hereinbelow with reference to FIGS. 6A-C.

For some applications, the computer processor identifies the tool that is currently disposed within the incision (i.e., which type of tool is currently disposed within the incision), and calculates a disposition of the remote center of motion location of the ophthalmic tool relative to the incision, based upon the tool that is identified as currently being disposed within the incision. For example, the computer processor identifies the tool that is currently disposed within the incision by analyzing images that are acquired using imaging system 22 (e.g., using machine vision algorithms). Alternatively or additionally, each of the tools may have a tool-identification component (e.g., a marker, a barcode, and/or a QR code), and the computer processor identifies the tool that is currently disposed within the incision by identifying the tool-identification component within images that are acquired using imaging system 22. For some applications, the computer processor is configured to receive a manual input identifying which tool is currently disposed within the incision. As described hereinabove, the computer processor typically drives the control-component unit to provide force feedback to the operator based on disposition of the remote center of motion location of the ophthalmic tool relative to the incision. Referring to FIG. 3B, for some applications, rather than constraining entry of the tool into the patient's eye to remain within an incision (which is typically only slightly larger than the maximum cross sectional dimension of the tool that passes through the incision point), entry of the tool into the patient's eye is constrained to remain within an incision zone 41, which is larger than an incision. For some applications, the area of the incision zone is more than 150 percent or more than 200 percent of the maximum cross sectional dimension of the tool that passes through the incision zone. For example, the entry of the tool into the patient's eye may be constrained to remain within an incision zone having an area of 2 mm² to 10 mm². Alternatively, the robotic system is configured to constrain entry of the tool into the patient's eye to remain within an incision that is only slightly larger than the maximum cross sectional dimension of the tool that passes through the incision, or to remain within an incision that is no larger than the maximum cross sectional dimension of the tool that passes through the incision.

As described hereinabove, typically, the robotic system is configured to assist the operator in moving tool 21, such that a tip of the tool is moved in a desired manner within the patient's eye, while entry of the tool into the patient's eye is maintained within the incision. The longitudinal portion of the tool that is within the incision and which functions as the remote center of motion is referred to herein as “the remote center of motion location of the tool”. (It is noted that over the course of the procedure, the location along the tool that is within the incision may change. The remote center of motion location of the tool refers to whichever location along the tool is currently within the incision.) In general, all descriptions of the robotic system assisting the operator in moving the tip of the tool within the patient's eye in such a manner that the remote center of motion location of the tool remains within the incision should be understood to either mean that the operator is assisted in maintaining the remote center of motion location of the tool within either the incision itself or within an incision zone that is larger than the incision by a predetermined amount (e.g., as described in the previous paragraph). For some applications, a force is applied to the operator via the control-component unit, with the force varying as a function of the distance of the outer edge of the tool relative to the center of the incision.

For some applications, the control-component unit is configured to apply a directional force that is calculated based upon the disposition (i.e., location and orientation) and movement of the control-component joysticks 30 and/or control-component tools 32. For example, the computer processor may perform velocity measurements on movement of the control-component tools and may calculate a force that is applied to the control-component arm that simulates physical interaction based upon the velocity measurements. Alternatively or additionally, the computer processor may perform measurements of the location of the ophthalmic tool relative the incision and may calculate a force that is applied to the control-component arm that simulates physical interaction based upon the location measurements. For some applications, the control-component arm is configured to apply torque to the user. For some applications, the feedback is configured to simulate a wall by applying force to the operator whenever they attempt to move a portion of the control-component tool 32 past a certain plane. For some such applications, the applied force is configured to be equal and opposite to the force applied to control-component tool 32 by the operator, such as to provide the sensation of a rigid wall that the operator cannot pass. Alternatively or additionally, the applied force is configured to be proportional to the distance of the outer edge of ophthalmic tool 21 from the center of the incision. Typically, this creates the sensation of an elastic, spring-like barrier, that is more difficult to enter the further it is penetrated.

Referring again to FIG. 3A, and purely by way of example, irrigation-aspiration tool 46 may be inserted via a 2.6 mm wide incision 40. The end of the irrigation-aspiration tool 46 has a well-defined longitudinal axis 52 that passes through its cross-section. It is typically desirable that longitudinal axis of irrigation-aspiration tool 46 be maintained as near to the center of incision 40 as possible. Assuming that the longitudinal axis of irrigation-aspiration tool 46 passes through the incision center, if the operator were to move the tool more than a given amount along the x axis, incision extension may occur. Typically, due to tissue flexibility it is possible to move the outer edge of the tool beyond the edge of the incision without causing corneal tearing. Therefore, as described hereinabove, for some applications, entry of the tool into the patient's eye is constrained to remain within an incision zone 41, which is larger than the incision.

For some applications, based on images of the tool and the patient's eye, as well as predetermined data regarding the tools dimensions, the computer processor is configured to determine the location and orientation of the remote center of motion location of the tool relative to the incision. For some applications, the computer processor determines the location of the incision based on the location and orientation of the keratome blade when the incision was made (as well as predetermined data regarding the width of the keratome blade), or by using computer vision, or a combination of the two. For some applications the computer processor determines the location of the tool's longitudinal axis relative to the incision (e.g., relative to the center of the incision, relative to an edge of the incision, and/or relative to the edge of an incision zone). In the case of some tools, the longitudinal axis is a straight line and the cross-section of the tool is symmetrical around its axis. In the case of some of the tools, the tool's longitudinal axis is not a straight line, but rather it differs at different locations along the length of the tool, with the longitudinal axis following the centroid of the tool's cross-section. For some applications, the computer processor determines the distance between the outer edge of the remote center of motion location of the tool relative to the incision (e.g., relative to the center of the incision, relative to an edge of the incision, and/or relative to the edge of an incision zone). Typically, the computer processor determines the magnitude and/or the direction of the feedback force that is provided to the operator based upon the above-mentioned calculations.

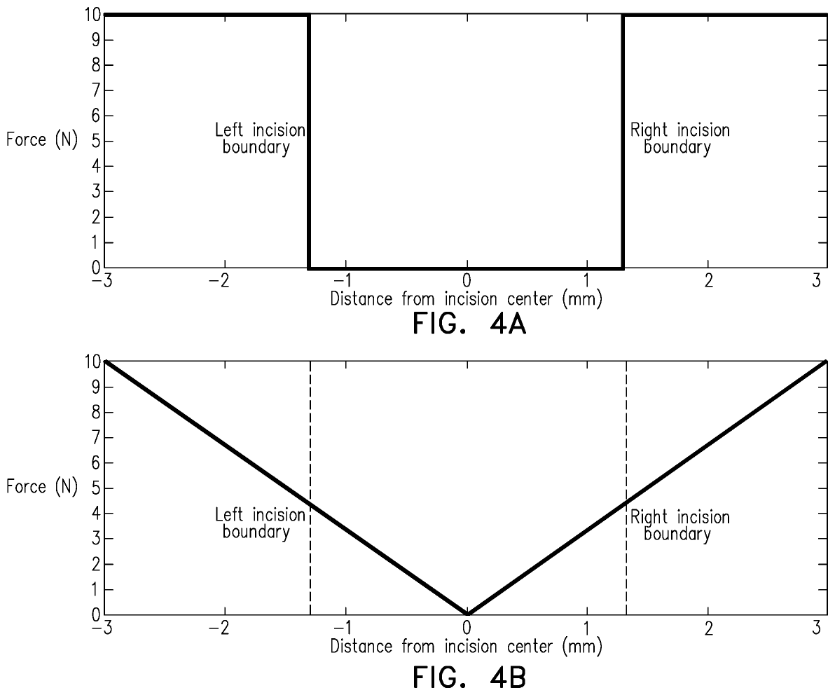

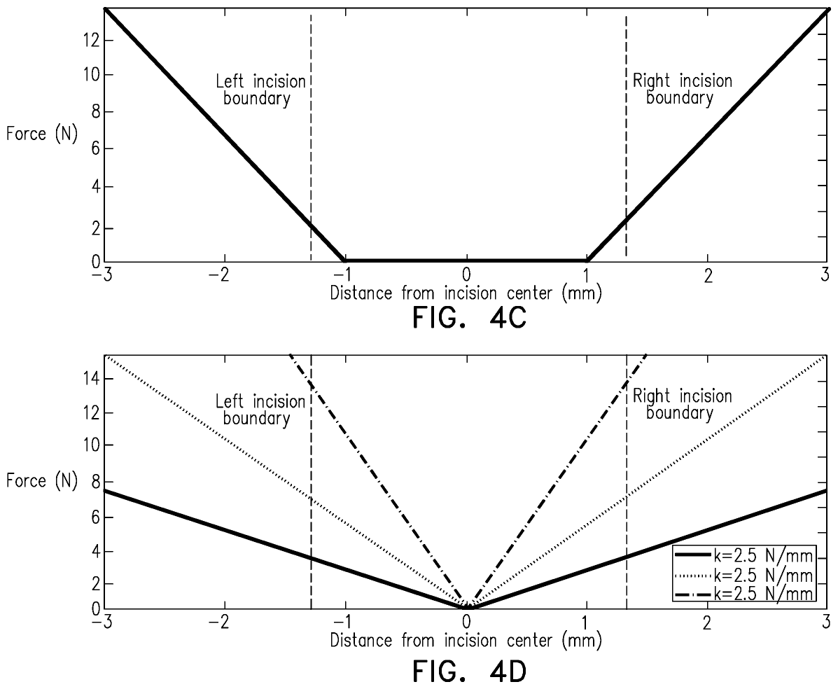

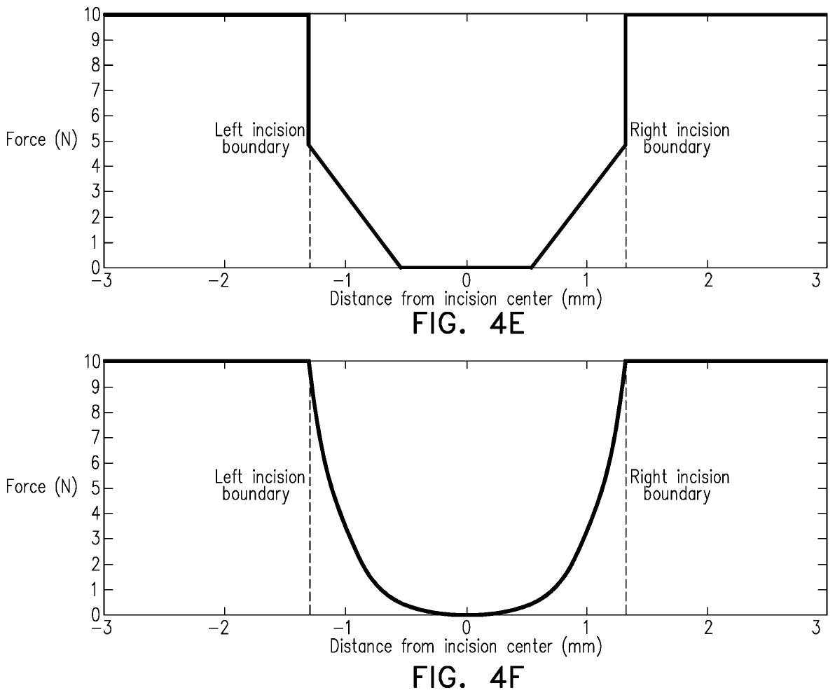

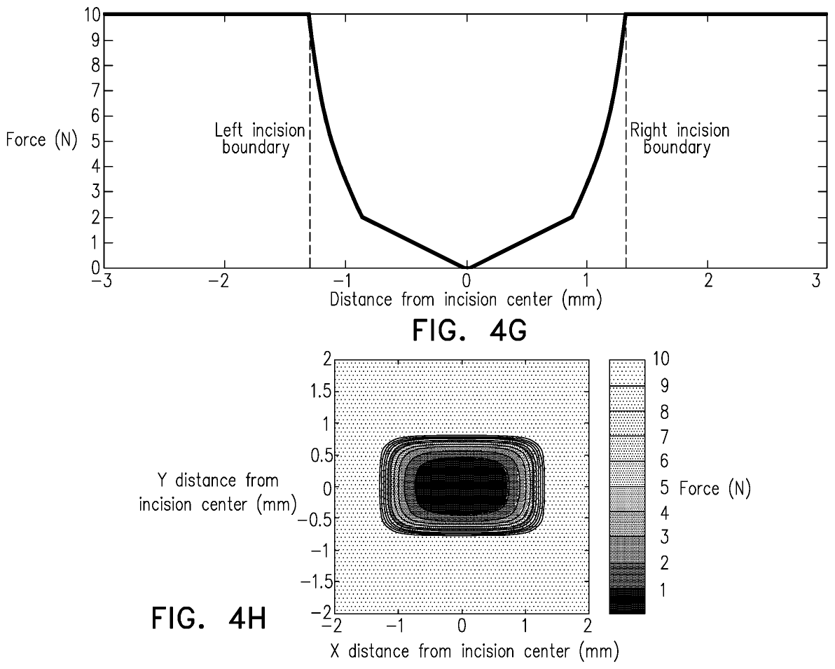

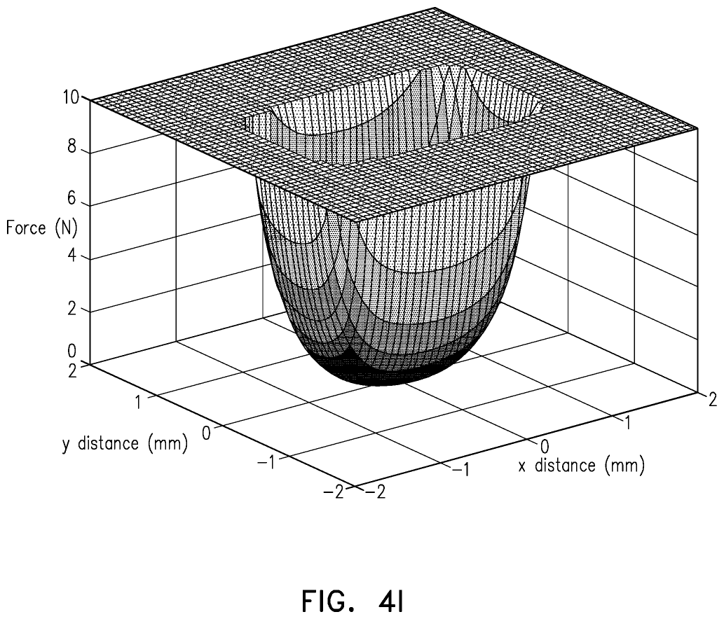

For some applications, based upon the above-mentioned calculations, the computer processor computes a force function, which returns a force vector that is to be provided by the control-component arm to the operator. The scope of the present disclosure includes providing any type of force functions, some of which are described in detail with reference to FIGS. 4A-I. For some applications, the force function is based on movement along the x axis, along the y axis, or both, with the force function in the two directions either being calculated as two independent functions, or as one function with two inputs. For some applications (e.g., in the case of a tool having a longitudinal axis that is a straight line and a constant cross section), force is not applied based on movement in along the z axis (i.e., retraction or advancement through the incision, with the z axis being perpendicular to the x and y axes) because movement along the z axis does not cause violation of the remote center of motion location of the tool remaining within the incision or within the incision zone. However, if the cross-section of the tool varies along its length, then movement along the z axis may result in feedback, since the location of the outer edge of the tool cross-section changes relative to the incision or the incision zone, as a result of movement along the z axis. Similarly, if the longitudinal axis of the tool is disposed at an angle with respect to the z axis, then movement along the z axis may result in feedback, since the location of the outer edge of the tool cross-section changes relative to the incision or the incision zone, as a result of movement along the z axis.

Reference is now made to FIGS. 4A, 4B, 4C, 4D, 4E, 4F, 4G, 4H, and 4I, which are graphs that graphically illustrate the variations in force that is applied to a control-component arm, as a function of the distance of an outer edge of a tool from an incision in a subject's cornea, in accordance with respective applications of the present invention.

Referring to FIG. 4A, for some applications, a step function is applied. As stated above with reference to FIG. 3A, by way of example, irrigation-aspiration tool 46 may be inserted via a 2.6 mm wide incision 40. In this example, a step function may be applied in the x direction, with an incision width of 2.6 mm:

F=0; |x|≤1.3 mm

F=10N; |x|>1.3 mm [Function 1]

In the present example, the force function is applied as a function of the distance of the longitudinal axis of the tool from the center of the incision at the remote center of motion location of the tool, although the scope of the present disclosure includes calculating the force function as a function of other variable, e.g., the distance of the outer edge of the tool from the outer edge of the incision at the remote center of motion location of the tool. In addition, the scope of the present disclosure should not be interpreted by the particular distances and forces provided in the above or the below examples. Rather, these examples are provided to demonstrate the type of force functions that may be provided. The scope of the present disclosure includes modifying these examples, such that these types of force functions are applied using different distances and forces to those provided.

Using Function 1, no force is applied when the operator moves the tool such that the tool's axis is less than 1.3 mm from the incision center in either direction along the x axis. When the operator moves the tool such that tool's axis is more than 1.3 mm from the incision center in either direction along the x axis, a force of 10 N is applied. In this example, only the force magnitude is shown. The force direction is typically opposite to the direction of violation, i.e. opposite of the sign of the distance. A more complete function is:

F=0; |x|≤1.3 mm

F=−10N; x>1.3 mm [Function 1]

F=+10N; x<1.3 mm [Function 2]

For simplicity, for all of the other functions that are described herein, the force is displayed as a magnitude. However, it should be understood that the direction of the force typically opposes the direction of motion and is directed towards the incision center.

A step function as shown in FIG. 4A acts to create a sensation of two virtual walls at the edges of the incision. The operator feels no force applied while the tool is within the incision, and 10 N of force (in the present example) opposing the operator's motion when trying to move the tool's axis more than 1.3 mm from the center of the incision. Typically, the force magnitude is made to be as high as the device will allow, to simulate a stiff wall. For such a force function, if the operator applies a force above 10 N (in the present example), the tool edge of the tool will move out of the incision, and the operator will feel a constant opposing force at the control-component arm.

Other options of force functions may be applied such as to generate a different sensation for the operator. One example is a linear function, as graphically shown in FIG. 4B. In such cases, the farther the operator moves the tool axis from the incision center (or the tool edge from the incision edge), the greater the force that is applied in an attempt to gravitate the tool back to the incision center. Such a function typically provides the operator with a “springy” sensation, which may reflect the feeling of a tool pushing against the edge of an incision more accurately than a step function. Furthermore, this allows the operator to push the tool beyond the edges of the incision, while providing the operator with a cue of the extent to which the tool has been pushed beyond the edges of the incision.

For some applications, a combination of functions is provided. For example, as graphically shown in FIG. 4C, a linear function may be applied within a certain distance from the incision center, whereas as the distance from the center exceeds a certain amount (1 mm in the example that is shown), a linear function is applied. In this case, the operator typically feels no resistance within the incision, but towards the edges of the incision, a force is applied that varies linearly with the distance from the incision center. This typically provides the operator some indication that are reaching or have reached the edges of the incision. As with the function graphically shown in FIG. 4B, this function allows the operator to push the tool beyond the edges of the incision, while providing the operator with a haptic cue of the extent to which the tool has been pushed beyond the edges of the incision

For some applications, parameters of the force function are configured to create a given sensation. For example, for a linear force function, one may change the stiffness k to change the feeling of the feedback, using Function 3 shown below:

F=k|x| [Function 3]

FIG. 4D graphically shows how the linear force function would look using different stiffness values, with each line representing a different stiffness value. Lower values of stiffness result in less force applied for a given distance from the incision center, and vice versa. For some applications, the robotic system allows the operator to select which force function they wish to be applied, and/or a level of stiffness that they wish to be applied (e.g., by providing an input to the computer processor), and to calculate the force to be applied to the operator at least partially based upon the operator's selection. Some operators may prefer a force function with a high stiffness (in order to receive clearer indication of the tool being moved toward or beyond the edges of the incision), while other operators may prefer a force function with a low stiffness (such that the extent to which they exert themselves against the force feedback is lower).

For some applications, different combinations of force functions are used. For example, as graphically shown in FIG. 4E, (a) when the tool's longitudinal axis is within a first given distance range from the incision center (0-0.5 mm in the example shown) no force is applied, (b) when the tool's axis within a second given distance range from the incision center (0.5 mm-1.3 mm in the example shown) a linear force function is applied, and (c) when the tool's axis within a third given distance range from the incision center (1.3 mm and greater, in the example shown), a step function is applied. For some applications, this combines advantages of each type of function, with the operator (a) not needing to exert themselves against the force feedback when near the incision center, (b) being provided with a gradual cue that they are approaching the incision edges, and (c) being provided with a “hard wall” sensation to prevent them from pushing the tool beyond the edge of the incision or the incision zone.

For some applications, other types of force functions are applied, e.g. an exponential force function, as graphically shown in FIG. 4F, which varies exponentially as follows:

F=b·e^{a|x|}+c [Function 4]

where a, b and c are configurable parameters, and e is Euler's number.

For some applications, such a function is configured to give the operator gradually increasing force feedback as they distance themselves from the center, creating a variable stiffness sensation. Other functions may also be used, such as functions that incorporate polynomials, logarithms, or powers.

For some applications additional combinations of functions are used. For example, as graphically shown in FIG. 4G, a linear function (closer to the center of the incision) may be combined with an exponential function (farther from the center of the incision).

FIGS. 4A-G graphically show functions that are applied based on displacement along the x axis. For some applications, similar functions are applied to displacement along the y axis. For some applications, the force to be applied based on displacement along the y axis is computed independently. Alternatively, a 2D force function is used based upon displacement along both the x axis and the y axis.

Purely by way of example, FIGS. 4H and 4I graphically show alternative representations of an example of a 2D exponential force function which are applied in accordance with some applications of the present invention.

For example, Function 5, presented below, may be used as the 2D force function:

F=b₁·^{a}^{₁}^{|x|}+b₂·e^{a}^{|y|}+c Function 5

The force output may be interpreted as a vector, or as a magnitude. If treated as a magnitude, the direction of the vector is typically toward the center of the incision.

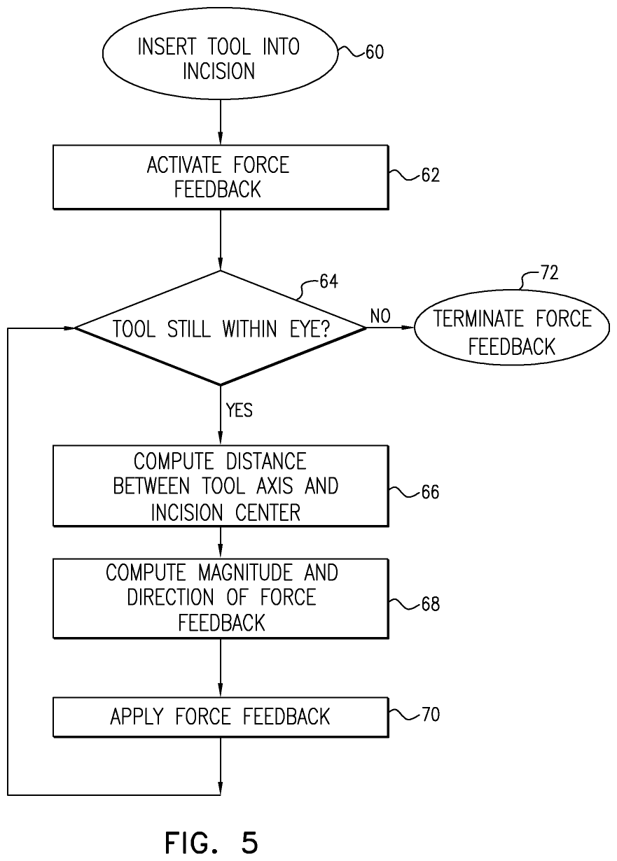

Reference is now made to FIG. 5, which is a flowchart showing steps of a procedure, in accordance with some applications of the present invention. In a first step 60, tool 21 is inserted into incision 40 (tool and incision shown in FIGS. 3A-B). In a second step 62, the force feedback functionality of the control-component unit is activated. In accordance with respective applications, the force feedback functionality of the control-component unit is activated automatically (in response to detecting that the tool has been inserted into the incision) or is activated manually by the operator. As described hereinabove, for some applications, the operator selects the type of force function and/or the stiffness that is to be used for the feedback.

Subsequently, the computer processor detects whether or not the tool is still inside the patient's eye (step 64). Assuming that the tool is still within the eye, the computer processor computes the distance between the tool's axis and the center of the incision, at the remote center of motion location along the tool (step 66). (As noted above, alternatively or additionally, the computer processor computes the distance between the edge of the tool and the edge of the incision or the end of an incision zone, at the remote center of motion location along the tool.) Based upon step 66, the computer processor computes the magnitude and the direction of the force that is to be provided to the operator by the control-component unit (step 68). In step 70, the force that was computed in step 68 is applied. Assuming that in step 64, it is detected that the tool is no longer in the eye, the force feedback is terminated (step 72). In accordance with respective applications, the force feedback functionality of the control-component unit is terminated automatically (in response to detecting that the tool has been removed from the incision) or is terminated manually by the operator.

Reference is now made to FIGS. 6A, 6B, and 6C, which are schematic illustrations of a joystick 30 and control-component tool 32 of a control-component unit 26, in accordance with some applications of the present invention. As indicated in FIGS. 6A, 6B, and 6C, for some applications joystick 30 is configured as a control-component arm that includes two or more links 80A, 80B, 80C that are connected via rotational arm joints 82A, 82B, 82C. The terms “joystick” and “control-component arm” are used interchangeably in the present disclosure. For some applications, a respective motor 84A, 84B, 84C is configured to control movement of each of the rotational arm joints, in order to provide feedback to the operator. Typically, the feedback is effective to cause a location 86 on control-component tool 32 to feel like a center of motion of the control-component tool, such that movement of the location is a given direction will provide a feedback force to the operator. Typically, the strength and direction of the feedback force will be in accordance with one of the examples described hereinabove. Further typically, the overall vector of the force will be composed of forces in x, y, and z directions of the control-component tool (indicated in FIG. 6A). It is noted that the x, y, and z directions of the control-component tool do not necessarily directly correspond to the x, y, and z direction of ophthalmic tool 21 (described hereinabove).

Referring to FIGS. 6B and 6C, it is noted that, typically, a majority of the motors (e.g., at least two of the motors (motors 84B and 84C)) directly apply torque to a rotational arm joint without requiring a gear or a belt to transfer the force, i.e., they are direct drive motors. For some applications, at least one of the motors (84A) applies torque to one of the rotational arm joints (82A) via a belt 88. Typically, a belt is used, such that the motor can be positioned closer to a base 90 of the control-component unit (base 90 being shown in FIG. 7), in order to reduce the weight and inertia that the operator feels, relative to if the third motor were to be placed closer to rotational arm joint 82A.

It is noted that, in order to increase the richness with which the feedback that the joystick provides to the operator reflects the position of the ophthalmic tool's remote center of motion location relative to the incision, it may be preferable to use a greater number of control-component motors. For example, for some applications, six motors are used, such that the control component is configured to apply a 3D force vector and a 3D torque vector. The scope of the present disclosure include using between one and six motors to provide feedback to the operator via the control component. However, using more than three motors typically adds additional weight and complexity to the design of the joystick. In addition, the inventors have found that using three motors provides a feedback that sufficiently reflects the position of the ophthalmic tool's remote center of motion location relative to the incision to be of assistance to the operator. Therefore, each of the joysticks typically includes three motors, as shown in FIGS. 6A and 6B.

Reference is now made to FIG. 7, which is a schematic illustration of some additional components of control-component unit 26, in accordance with some applications of the present invention. Typically, in addition to the above-described motors, each of the control-component arms includes a respective rotary encoder 92 coupled to each one of the three rotational arm joints 82A, 82B, 82C (e.g., as described in U.S. patent application Ser. No. 17/818,477, which is a continuation of WO 22-023962 to Glozman, which is incorporated herein by reference). The rotary encoders are configured to detect movement of the respective rotational arm joints and to generate rotary-encoder data in response thereto. For some applications, the control-component arm additionally includes an inertial-measurement unit 94 that includes a three-axis accelerometer, a three-axis gyroscope, and/or a three-axis magnetometer. The rotary encoders and inertial-measurement unit are collectively referred to herein as “location sensors”. The inertial-measurement unit typically generates inertial-measurement-unit data relating to a three-dimensional orientation of the control-component arm, in response to the control-component arm being moved. For some applications, computer processor 28 receives the rotary-encoder data and the inertial-measurement-unit data. Typically, the computer processor determines the XYZ location of the tip of the control-component tool 32, based upon the rotary-encoder data, and determines the orientation of the tip of control-component tool 32 (e.g., the 3 Euler angles of orientation, and/or another representation of orientation) based upon the inertial-measurement-unit data, or based upon a combination of the rotary-encoder data and the inertial-measurement-unit data. Thus, based upon the combination of the rotary-encoder data and the inertial-measurement-unit data, the computer processor is configured to determine the XYZ location and orientation of the tip of the control-component tool.

For some applications, the computer processor drives the robotic unit such that the tip of the ophthalmic tool that is being used to perform the procedure tracks the movements of the tip of the control-component tool. For some applications, the computer processor drives the robotic unit such that the tip of the ophthalmic tool that is being used to perform the procedure tracks the movements of the tip of the control-component tool in six degrees-of-freedom. Typically, incorporating an inertial-measurement unit to detect the three-dimensional orientation of the control-component arm allows the operator to control movement of the robotic unit using a reduced number of sensors, relative to if rotary encoders were used to detect motion of the control-component arm in all six degrees-of-freedom. Further typically, reducing the number of rotary encoders that are used tends to reduce the overall complexity of the control-component arm, since introducing additional rotary encoders would require additional wires to pass through rotating joints.

Notwithstanding the complexity associated with having additional rotary encoders, for some applications, the control-component arm includes more than three rotary encoders as well as an inertial-measurement unit, for redundancy, i.e., such that there are additional location sensors that may be used by the system in the event that some of the location sensors fail. For some such applications, the control-component arm includes an additional rotary encoder at each of the rotational arm joints, for redundancy. In addition, for some applications, the control component includes rotary encoders to detect the roll, pitch and yaw of tool 32 of the control-component tool, in addition to the inertial-measurement unit, for redundancy. For some such applications, tool 32 is coupled to the control-component arm via three rotational tool joints, corresponding to the roll, pitch and yaw of tool 32. Typically, the aforementioned rotary encoders detect motion of respective rotational tool joints via which the control-component tool is coupled to the control-component arm.

Although some applications of the present invention are described with reference to cataract surgery, the scope of the present application includes applying the apparatus and methods described herein to other medical procedures, mutatis mutandis. In particular, the apparatus and methods described herein to other medical procedures may be applied to other microsurgical procedures, such as general surgery, orthopedic surgery, gynecological surgery, otolaryngology, neurosurgery, oral and maxillofacial surgery, plastic surgery, podiatric surgery, vascular surgery, and/or pediatric surgery that is performed using microsurgical techniques. For some such applications, the imaging system includes one or more microscopic imaging units.

It is noted that the scope of the present application includes applying the apparatus and methods described herein to intraocular procedures, other than cataract surgery, mutatis mutandis. Such procedures may include collagen crosslinking, endothelial keratoplasty (e.g., DSEK, DMEK, and/or PDEK), DSO (descemet stripping without transplantation), laser assisted keratoplasty, keratoplasty, LASIK/PRK, SMILE, pterygium, ocular surface cancer treatment, secondary IOL placement (sutured, transconjunctival, etc.), iris repair, IOL reposition, IOL exchange, superficial keratectomy, Minimally Invasive Glaucoma Surgery (MIGS), limbal stem cell transplantation, astigmatic keratotomy, Limbal Relaxing Incisions (LRI), amniotic membrane transplantation (AMT), glaucoma surgery (e.g., trabs, tubes, minimally invasive glaucoma surgery), automated lamellar keratoplasty (ALK), anterior vitrectomy, and/or pars plana anterior vitrectomy.

Applications of the invention described herein can take the form of a computer program product accessible from a computer-usable or computer-readable medium (e.g., a non-transitory computer-readable medium) providing program code for use by or in connection with a computer or any instruction execution system, such as computer processor 28. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W), DVD, and a USB drive.

A data processing system suitable for storing and/or executing program code will include at least one processor (e.g., computer processor 28) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that the algorithms described herein, can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 28) or other programmable data processing apparatus, create means for implementing the functions/acts specified in the algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the algorithms described in the present application.

Computer processor 28 is typically a hardware device programmed with computer program instructions to produce a special purpose computer. For example, when programmed to perform the algorithms described with reference to the Figures, computer processor 28 typically acts as a special purpose robotic-system computer processor. Typically, the operations described herein that are performed by computer processor 28 transform the physical state of a memory, which is a real physical article, to have a different magnetic polarity, electrical charge, or the like depending on the technology of the memory that is used. For some applications, operations that are described as being performed by a computer processor are performed by a plurality of computer processors in combination with each other.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An apparatus for performing a procedure on an eye of a patient using an ophthalmic tool that has a tip, the apparatus comprising:
a robotic unit configured to move the tool;
a control-component unit that comprises:
a control-component tool that is configured to be moved by an operator and that defines a tip; and
a control-component arm coupled to the control-component tool and comprising:
a plurality of links that are coupled to each other via rotational arm joints;
one or more location sensors; and
one or more motors that are operatively coupled to respective rotational arm joints:
a computer processor configured to:
drive the robotic unit to insert the ophthalmic tool into the patient's eye via an incision in a cornea of the patient's eye, such that a tip of the ophthalmic tool is disposed within the patient's eye;
determine a location and orientation of the tip of the control-component tool based upon data received from the one or more locations sensors;
move the tip of the selected ophthalmic tool within the patient's eye in a manner that corresponds with movement of the control-component tool; and
provide force feedback to the operator by driving the control-component arm using the plurality of motors.

2. The apparatus according to claim 1, wherein the control component comprises exactly three motors operatively coupled to respective rotational arm joints.

3. The apparatus according to claim 1, wherein the control-component arm comprises a belt, and at least one of the motors is operatively coupled to a corresponding one of the rotational arm joints via the belt, such that the at least one of the motors is disposed closer to a base of the control-component unit than if the at least one of the motors directly drove the corresponding one of the rotational arm joints.

4. The apparatus according to claim 1, wherein a majority of the one or more motors directly drive a corresponding one of the rotational arm joints to which they are operatively coupled.

5. The apparatus according to claim 1, wherein the one or more location sensors comprise:
three rotary encoders, each of the three rotary encoders coupled to a respective one of the rotational arm joints and configured to detect movement of the respective rotational arm joint and to generate rotary-encoder data indicative of an XYZ location of the tip of the control-component tool in response thereto; and
an inertial measurement unit comprising at least one of sensor selected from the group consisting of: a three-axis accelerometer, a three-axis gyroscope, and a three-axis magnetometer, the inertial measurement unit being configured to generate inertial-measurement-unit data indicative of an orientation of the tip of control-component tool.

6. The apparatus according to claim 1, wherein the control-component tool is coupled to the control-component arm via three rotational tool joints, and wherein the one or more location sensors comprise:
two rotary encoders coupled to each one of the rotational arm joints and configured to detect movement of the rotational arm joint and to generate rotary-encoder data indicative of an XYZ location of the tip of the control-component tool, in response thereto; and
one rotary encoder coupled to each one of the rotational tool joints and configured to detect movement of the rotational tool joint and to generate rotary-encoder data indicative of an orientation of the tip of the control-component tool, in response thereto;
an inertial measurement unit comprising at least one of sensor selected from the group consisting of: a three-axis accelerometer, a three-axis gyroscope, and a three-axis magnetometer, the inertial measurement unit being configured to generate inertial-measurement-unit data indicative of an orientation of the tip of control-component tool.

7. The apparatus according to claim 1, wherein the computer processor is configured to:
drive the robotic unit to insert the ophthalmic tool into the patient's eye via the incision in the cornea of the patient's eye, such that the tip of the ophthalmic tool is disposed within the patient's eye and a remote center of motion location of the ophthalmic tool is disposed within the incision; and
provide force feedback to the operator that is indicative of a disposition of the remote center of motion location of the ophthalmic tool relative to the incision.

8. The apparatus according to claim 7, wherein the computer processor is configured to:
determine an identity of the ophthalmic tool that has been inserted into the patient's eye, and
based upon the identity of the ophthalmic tool, calculate a disposition of the remote center of motion location of the ophthalmic tool relative to the incision.

9. The apparatus according to claim 7, wherein the computer processor is configured to provide force feedback to the operator via the control component, by:
performing velocity measurements on the control-component tool,
calculating a force to be applied to the operator based on the velocity measurements, and
driving the control component to apply the calculated force to the operator, via the one or more motors.

10. The apparatus according to claim 7, wherein the computer processor is configured to provide force feedback to the operator via the control component, by:
performing measurements of a position of the ophthalmic tool relative to the incision,
calculating a force to be applied to the operator based on the position measurements, and
driving the control component to apply the calculated force to the operator.

11. The apparatus according to claim 7, wherein the computer processor is configured to calculate a force to be applied to the operator by calculating the force such as to be equal and opposite to a force applied to the control-component tool by the operator.

12. The apparatus according to claim 7, wherein the computer processor is configured to calculate a force to be applied to the operator by calculating the force such as to be proportional to a distance of an outer edge of the ophthalmic tool from a center of the incision.

13. The apparatus according to claim 7, wherein the computer processor is configured to receive an input from the operator that is indicative of a stiffness of force feedback that they wish to receive, and to calculate a force to be applied to the operator at least partially based upon the input from the operator.

14. The apparatus according to claim 7, wherein the computer processor is configured to constrain movement of the control-component tool in a manner that corresponds to how movement of the remote center of motion location of the ophthalmic tool relative to the incision should be constrained.

15. The apparatus according to claim 14, wherein the computer processor is configured to constrain movement of the control-component tool in a manner that constrains the remote center of motion location of the ophthalmic tool to remain within an incision zone that is larger than the incision.

16. The apparatus according to claim 14, wherein the computer processor is configured to constrain movement of the control-component tool in a manner that constrains the remote center of motion location of the ophthalmic tool to remain within the incision.

17. The apparatus according to claim 7, wherein the computer processor is configured to calculate a force to be applied to the operator by calculating a force function that is based on a distance of an outer edge of the ophthalmic tool from a center of the incision in two directions.

18. The apparatus according to claim 17, wherein a first one of the two directions is parallel to the incision and at a tangent to the cornea of the patient's eye at the incision, and a second one of the two directions is normal to the first direction and at a tangent to the cornea of the patient's eye at the incision.

19. A method for performing a procedure on an eye of a patient using an ophthalmic tool that has a tip, the method comprising:
driving a robotic unit to insert the ophthalmic tool into the patient's eye via an incision in a cornea of the patient's eye, such that a tip of the ophthalmic tool is disposed within the patient's eye and a remote center of motion location of the ophthalmic tool is disposed within the incision;
determining the location and the orientation of the tip of a control-component tool that is configured to be moved by an operator, based upon data received from one or more location sensors that are disposed on a control-component arm that is coupled to the control-component tool;
moving the tip of the ophthalmic tool within the patient's eye in a manner that corresponds with movement of the control-component tool; and
providing force feedback to the operator via the control-component arm, wherein the control component arm includes a plurality of links that are coupled to each other via rotational arm joints and one or more motors that are operatively coupled to respective rotational arm joints and the force feedback is provided to the operator by driving the control-component arm using the plurality of motors.
